(19) United States
(12) Patent Application Publication
(10) Pub. No.: US 2026/0053726 A1
(43) Pub. Date: Feb. 26, 2026

(54) ODOR ELIMINATING COMPOSITION

(71) Applicant: Anthony Cucetta, Williamstown, NJ (US)

(72) Inventor: Anthony Cucetta, Williamstown, NJ (US)

(21) Appl. No.: 18/809,458

(22) Filed: Aug. 20, 2024

(51) Int. Cl. A61K 8/92; A61K 8/64; A61K 8/67; A61L 2/00; A61L 2/18; A61L 101/32; A61Q 5/02

(52) U.S. Cl. CPC A61K 8/922; A61K 8/64; A61K 8/676; A61K 8/678; A61L 2/0088; A61L 2/18; A61Q 5/02; A61L 2101/32

(57) ABSTRACT

A composition for eliminating the odor of animal secretions of sprayed animals and articles can, in one embodiment, include garlic oil and reduced glutathione. A method and kit for eliminating the odor of animal secretions of sprayed animals and articles using the odor eliminating composition can optionally include 13% w/v to 25% w/v one or more of a surfactant, 1% w/v to 11% w/v one or more of an emulsifier, 1.0% w/v to 5.0% w/v one or more of a humectant, 1.0% w/v 10.0% w/v one or more of an oil, 1.0% w/v to 6.0% w/v one or more of a preservative, 0.1% w/v to 5.0% w/v one or more of an antioxidant, 0.1% w/v to 1.5% w/v one or more of a pH stabilizer, and 40% w/v to 85% w/v one or more of a solvent.

### FIELD

The present disclosure generally relates to odor eliminating compositions.

### BACKGROUND

Humans have long attempted to mask and/or eliminate odors that they encounter or create.

Certain animals, for example, may utilize a defensive mechanism wherein the animal emits one or more secretions to deter predators and provide protection from possible harm. One of the most well-known animals utilizing such a mechanism is the skunk, a mammal in the family Mephitidae. The skunk is capable of spraying a strong, malodorous odor from its anal glands. Humans and domesticated animals such as dogs and cats often encounter the odor at least once. If left untreated, the odor is capable of remaining odorous for 14 to 21 days.

Thiols, also known as Mercaptan, thioacetates, and other organic sulfur compounds are responsible for emitting the odor from the skunk's spray. The conventional method of odor elimination using tomato juice has been found to be ineffective. Instead of eliminating the odor, tomato juice simply results in olfactory fatigue wherein the nose is overwhelmed by the odor and adapts to the smell. Conventional methods including other ingredients such as baking soda, hydrogen peroxide, and/or vinegar may be toxic to dogs if ingested or may result in skin irritation. A prior art skunk odor shampoo is disclosed in U.S. Pat. No. 4,834,901, which is hereby incorporated by reference. Washing the animal with soap and water may result in a stronger odor, as dish soap cannot eliminate odor-causing thiols.

### SUMMARY

In light of the above-outlined deficiencies of the prior art, effective, safe, and/or organic odor eliminating compositions would be desirable.

The odor eliminating composition of the present disclosure overcomes the challenges in the prior art.

The present disclosure provides methods and kits for eliminating or at least reducing order, such as the odor of animal secretions from sprayed animals and articles.

In one embodiment, the odor eliminating composition includes 13% weight/volume (w/v) to 25% w/v one or more of a surfactant, 1% w/v to 11% w/v one or more of an emulsifier, 1.0% w/v to 5.0% w/v one or more of a humectant, 1.0% w/v 10.0% w/v one or more of an oil, 1.0% w/v to 6.0% w/v one or more of a preservative, 0.1% w/v to 5.0% w/v one or more of an antioxidant, 0.1% w/v to 1.5% w/v one or more of a pH stabilizer, and 40% w/v to 85% w/v one or more of a solvent, wherein the one or more of an antioxidant includes reduced glutathione, and wherein the one or more of an oil comprises garlic oil.

### DETAILED DESCRIPTION

In one embodiment, the present disclosure provides an odor eliminating composition to effectively eliminate the odor of secretions emitted by animals, including, but not limited to, a skunk, other animals in the family Mephitidae, mink, weasel, other animals in the family Mustelidae, possum, other animals in the Didelphidae family, and any other mammal known in the art to release a secretion having an unpleasant odor to the human senses.

Thiols, thioacetates, and other organic sulfur compounds are responsible for the odor from the secretions. The odor eliminating composition of the present disclosure is capable of degrading thiols to eliminate their odor, including, but not limited to, (E)-2-butenyl 3-methylbutyl disulfide, bis(3-methylbutyl) disulfide, bis[(E)-2-butenyl] disulfide, 2-quinolinemethanethiol, phenylmethanethiol, 2-methylquinoline, any combination thereof, and the like.

The human nose may easily ascertain the odor from a skunk in the air, even at concentrations as low as 10 parts per billion. About 5 mL to 10 mL of thiols and organosulfur compounds are released from the Skunk's anal glands when a skunk sprays an animal. When the secretions are sprayed on a domesticated animal, humans are faced with the arduous task of cleaning the animal and trying to restore the animal to its normal scent. Conventional odor eliminating compositions or methods are ineffective and/or harmful. As a non-limiting example, there is a long-established misconception that tomato juice eliminates skunk odor; however, tomato juice simply results in olfactory fatigue wherein the nose is overwhelmed by the odor and adapts to the smell. Many odor eliminating compositions known in the art include one or more components harmful to animals. As a non-limiting example, conventional odor eliminating compositions may include at least one of baking soda, hydrogen peroxide, vinegar, potassium iodate, lithium chloride, solanine, sodium hypochlorite, calcium hypochlorite, ammonia, phenols, formaldehyde, phthalates, acrylamide, polyquarternium-10, propylene glycol, methylchloroisothiazolinone, Methylisothiazolinone, PEG 8 laurate, laureth-4, 1,4-dioxane, coloring agents (e.g., Red #40, Yellow #5, Blue #1, and the like), benzene, peppermint, potassium sorbate, and the like, which may cause skin irritation, vomiting, drooling, runny eyes, rashes, collapse, burns, blood disorders, respiratory distress, respiratory irritation, genotoxic effects, chromosomal aberrations, cytotoxic effects, hormonal imbalance, digestive irritation, central nervous system issues (e.g., seizures, paralysis, lack of coordination, weakness, etc.), allergic reaction, organ failure, cancer and the like. The conventional method of cleaning the animal with soap and/or water is ineffective, as it may cause the odor to remain the same or increase, as conventional soap cannot break the sulfur-hydrogen bonds within odor-causing thiols.

The odor eliminating composition described herein addresses one or more of the shortcomings known from conventional methods and compositions by providing an effective, safe, natural, and/or organic composition.

The thiols and organosulfur compounds of skunk secretions may be readily oxidized into sulfides, sulfoxides, sulphones, and sulphates. The oxidized products are odorless or substantially less malodourous compared to the unreacted thiols and organosulfur compounds. The oxidized products are also soluble in water, allowing the products to be easily removed via the odor eliminating composition.

The odor eliminating composition of one embodiment of the presently disclosed technology may include garlic oil and L-glutathione to break down the odor-causing thiols and organosulfur compounds. The odor eliminating composition may optionally include Vitamin C.

In conventional compositions and cosmetics, garlic oil is not successfully used because it retains a garlic smell and users typically do not want to have a garlic odor. The present disclosure provides a novel combination of garlic oil and L-glutathione to eliminate thiols and organic sulfur compounds present in animal secretions. The amount of garlic oil present in the composition may be sufficient to enable the chemical reactions necessary to break down the thiols and organic sulfur compounds but insufficient to cause an uncomfortable smell for the animal or ordinary person with ordinary olfactory senses.

Garlic oil may include sulfur compounds, enzymes, minerals and amino acids, including, but not limited to, aliin, allicin (diallyl thiosulphate or 2-propene-1-sulfinothioc acid s-2-propenyl ester), diallyl sulfide, diallyl disulfide, diallyl trisulfide, S-allylcysteine, S-allylmercaptocystein, allinase, peroxidases, and myrosinase, arginine, selenium, germanium, tellurium, and the like.

The garlic oil and L-glutathione may synergistically interact with thiols and organic sulfur compounds present in skunk secretions to eliminate the odor. As a non-limiting example, diallyl trisulfide of garlic oil may react with L-glutathione to form oxidized glutathione and release allyl thiol. Oxidized glutathione may be reduced using Vitamin C (ascorbic acid) to L-glutathione to ensure a continuous reaction and release of allyl thiol. Allyl thiol may react with the thiols and organosulfur compounds present in the skunk secretions, wherein a thiol-disulfide exchange may occur to form a new disulfide bond and regenerate L-glutathione. As a further non-limiting example, S-allylcysteine of garlic oil may react with L-glutathione to release allyl thiol and form oxidized glutathione. The oxidized glutathione may be reduced via Vitamin C to L-glutathione to ensure a continuous reaction and release of allyl thiol. The reaction may occur in water, which may ionize Vitamin C and L-glutathione, thereby increasing reactivity. The allyl thiol may react with the thiols and organosulfur compounds present in the skunk secretions, wherein a thiol-disulfide exchange may occur to form a new disulfide bond and regenerate L-glutathione. As a further non-limiting example, diallyl disulfide of garlic oil may react with L-glutathione to release allyl thiol and form oxidized glutathione. The oxidized glutathione may be reduced via Vitamin C to L-glutathione to ensure a continuous reaction and release of allyl thiol. The reaction may occur in water, which may ionize Vitamin C and L-glutathione, thereby increasing reactivity. The allyl thiol may react with the thiols and organosulfur compounds present in the skunk secretions, wherein a thiol-disulfide exchange may occur to form a new disulfide bond and regenerate L-glutathione.

While Vitamin C is discussed in the reactions and odor elimination composition of the present disclosure, Vitamin C is not required for the reactions to occur or for the odor to be eliminated. Vitamin C may be used to provide an increased supply of oxidized glutathione and reduced glutathione through reduction-oxidation reactions.

While garlic oil is described herein, other compounds are possible and within the scope of the present disclosure. Individual, isolated components of garlic oil may be added to the odor elimination composition, including, but not limited to, diallyl trisulfide, diallyl disulfide, S-allylcysteine, any combination thereof, and the like. Other compounds capable of reacting with L-glutathione or a L-glutathione equivalent to release allyl thiol or an allyl thiol equivalent are possible and within the scope of the present disclosure.

As used herein, “L-glutathione” and reduced glutathione may be used interchangeably. While L-glutathione is described herein, other compounds capable of reacting with another compound to release allyl thiol or an allyl thiol equivalent are possible and within the scope of the present disclosure.

While Vitamin C or ascorbic acid is described herein, other compounds or additional compounds capable of reducing oxidized glutathione or any L-glutathione equivalent are possible and within the scope of the present disclosure. As a non-limiting example, Vitamin E may be combined with Vitamin C to increase the reaction strength or reactivity. Vitamin C may act as a potent electron donor and/or reducing agent, while Vitamin E may act as an electron donor.

In one embodiment, the odor eliminating composition of the presently disclosed technology may include one or more surfactants. As a non-limiting example, the odor eliminating composition may include at least 1, 2, 3, 4, 5, 6, 7, 8, 9, and at least 10 surfactants. Surfactants may include any compound that reduces the surface tension of liquids, including, but not limited to, decyl glucoside, cocamidopropyl betaine, coco betaine, coco glucoside, decal glucoside, sucrose cocoate, capryl/capryl glucoside, lauryl glucoside, saponified coconut oil, potassium cocoate, any combination thereof, and other natural surfactants known in the art.

In one embodiment, the odor eliminating composition of the presently disclosed technology may include synthetic surfactants known to be safe for use on animals. As used herein, surfactants may include a soap, including any compound produced from the hydrolysis of fats in a saponification reaction. Soaps may include, but are not limited to, any soap made from the reaction of an acid and a base to form a salt, wherein the acid may include saturated (myristic acid, lauric acid, palmitic acid, stearic acid, and the like) and unsaturated fatty acids (oleic acid, linoleic acid, linolenic acid, ricinoleic acid, and the lake), and wherein the base may include sodium hydroxide, potassium hydroxide, potassium carbonate, and the like.

In one embodiment, the one or more surfactants may be present in at least 13% w/v to 25% w/v. The one or more surfactants may be at least 13% w/v, including, but not limited to, 13, 14, 15, 16, 17, 18, 19, 20, 21, 22, 23, 24, and at least 25% w/v. The one or more surfactants may be no more than 25% w/v, including, but not limited to, no more than 25, 24, 23, 22, 21, 20, 19, 18, 17, 16, 15, 14 and no more than 13% w/v. Any combination of the lower and upper limits is within the scope of the present disclosure, including, but not limited to, 13% w/v to 16% w/v, 13% w/v to 18% w/v, 14% w/v to 21% w/v, 17% w/v to 19% w/v, 17% w/v to 21% w/v, 19% w/v to 21% w/v, 17% w/v to 23% w/v, and 16% w/v to 24% w/v.

The odor eliminating composition of the presently disclosed technology may include one or more emulsifiers. As a non-limiting example, the odor eliminating composition may include at least 1, 2, 3, 4, 5, 6, 7, 8, 9, and at least 10 emulsifiers. Emulsifiers may include any compound capable of acting and/or configured to act as a stabilizer for oil dispersion in water, including, but not limited to, cetearyl wheat straw glucosides, cetearyl alcohol, glyceryl stearate, sodium stearoyl lactylate, sorbitan olivate, vegetable glycerin, sorbitan olivate, sunflower lecithin, emulsifier lecithin, any combination thereof, and the like.

Optionally, the one or more emulsifiers may be present in at least 1% w/v to 11% w/v. The one or more emulsifiers may be at least 1% w/v, including, but not limited to, 1, 2, 3, 4, 5, 6, 7, 8, 9, 10, and at least 11% w/v. The one or more emulsifiers may be no more than 11% w/v, including, but not limited to, no more than 11, 10, 9, 8, 7, 6, 5, 4, 3, 2, and no more than 1% w/v. Any combination of the lower and upper limits is within the scope of the present disclosure, including, but not limited to, 1% w/v to 5% w/v, 4% w/v to 8% w/v, 5% w/v to 7% w/v, 5% w/v to 9% w/v, 5% w/v to 10% w/v, and 4% w/v 10% w/v.

In one embodiment, the odor eliminating composition of the presently disclosed technology may include one or more humectants. As a non-limiting example, the odor eliminating composition may include at least 1, 2, 3, 4, 5, 6, 7, 8, 9, and at least 10 humectants. Humectants may include any substance that attracts water and may be used to keep things moist, including, but not limited to, glycerin, urea, hyaluronic acid, salicylic acid, xanthan gum, aloe vera, aloe vera oil, alpha hydroxy acids (glycolic acid, lactic acid, and the like), honey, sorbitol, any combination thereof, and the like.

Optionally, the one or more humectants may be present in at least 1.0% w/v to 5.0% w/v. The one or more humectants may be at least 1% w/v, including, but not limited to, 1.0, 1.2, 1.4, 1.6, 1.8, 2.0, 2.2, 2.4, 2.6, 2.8, 3.0, 3.5, 4.0, 4.5 and at least 5.0% w/v. The one or more humectants may be no more than 5.0% w/v, including, but not limited to, no more than 5.0, 4.5, 4.0, 3.5, 3.0, 2.8, 2.6, 2.4, 2.2, 2.0, 1.8, 1.6, 1.4, 1.2, and no more than 1.0% w/v. Any combination of the lower and upper limits is within the scope of the present disclosure, including, but not limited to, 1.0% w/v to 1.4% w/v, 1.0% w/v to 2.0% w/v, 1.2% w/v to 2.0% w/v, 1.4% w/v to 1.8% w/v, 1.4% w/v to 2.4% w/v, 1.4% w/v to 2.8% w/v, 1.2% w/v to 4.5% w/v, and 1.6% w/v 2.0% w/v.

In one embodiment, the odor eliminating composition of the presently disclosed technology may include one or more oils. As a non-limiting example, the odor eliminating composition may include at least 1, 2, 3, 4, 5, 6, 7, 8, 9, 10, 11, 12, 13, 14, and at least 15 oils. As used herein, “oils” may include “essential oils”. An essential oil includes any natural, volatile, and aromatic compound obtained from plant material (e.g., flowers, buds, seeds, leaves, herbs, wood, fruit, roots, and/or the like), including, but not limited to ginger oil, lemongrass oil, lemon oil, orange oil, lavender oil, chamomile oil, myrrh oil, frankincense oil, rosemary oil, bergamot oil, any combination thereof, and/or the like. An oil may include any nonpolar substance including hydrocarbons that has at least some hydrophobic and lipophilic properties, including, but not limited to, garlic oil, coconut oil, shea nut oil, grape-seed oil, hempseed oil, jojoba oil, seed oils, fruit extracts, any combination thereof, and/or the like.

Optionally, the one or more oils may be present in at least 1.0% to 10.0% w/v. The one or more oils may be at least 1.0% w/v, including, but not limited to, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 and at least 10.0% w/v. The one or more oils may be no more than 10.0% w/v, including, but not limited to, no more than 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, and no more than 1.0% w/v. Any combination of the lower and upper limits is within the scope of the present disclosure, including, but not limited to, 1.0% w/v to 7% w/v, 2.5% w/v to 7.5% w/v, 4.0% w/v to 6.0% w/v, 5.0% w/v to 6.0% w/v, 4.0% w/v to 10.0% w/v, 6.0% w/v to 8.0% w/v, and 3.0% w/v 8.0% w/v.

Optionally, the one or more oils may include 0.005% w/v to 0.040% w/v garlic oil. The garlic oil may include at least 0.005% w/v, including, but not limited to, at least 0.005, 0.0075, 0.010, 0.0125, 0.015, 0.0175, 0.020, 0.025, 0.030, 0.035, and at least 0.040% w/v garlic oil. The garlic oil may include no more than 0.040% w/v garlic oil, including, but not limited to, 0.040, 0.035, 0.030, 0.025, 0.020, 0.0175, 0.015, 0.0125, 0.010, 0.0075, and no more than 0.005% w/v. Any combination of the lower and upper limits is within the scope of the present disclosure, including, but not limited to, 0.005% w/v to 0.0175% w/v, 0.0125% w/v to 0.0175% w/v, 0.0125% w/v to 0.015% w/v, 0.015% w/v to 0.0175% w/v, 0.0125% w/v to 0.030% w/v, and 0.010% w/v to 0.020% w/v.

In one embodiment, the odor eliminating composition of the presently disclosed technology may include one or more preservatives. As a non-limiting example, the odor eliminating composition may include at least 1, 2, 3, 4, 5, 6, 7, 8, 9, and at least 10 preservatives. A preservative may include any compound capable of protecting from contamination (microbial, yeast, mold, and the like) and/or increasing the shelf-life of the odor eliminating composition, including, but not limited to, potassium sorbate, ground rosemary, apple cider vinegar, sodium benzoate, alcohol, glycerin, grapefruit seed extract, triethyl citrate, benzyl alcohol, tocopherol, and the like.

Optionally, the one or more preservatives may be present in at least 1.0% to 6.0% w/v. The one or more preservatives may be at least 1.0% w/v, including, but not limited to, 1.0, 1.5, 2.0, 2.5, 3.0, 3.5, 4.0, 4.5, 5.0, 5.5, and at least 6.0% w/v. The one or more preservatives may be no more than 6.0% w/v, including, but not limited to, no more than 6.0, 5.5, 5.0, 4.5, 4.0, 3.5, 3.0, 2.5, 2.0, 1.5, and no more than 1.0% w/v. Any combination of the lower and upper limits is within the scope of the present disclosure, including, but not limited to, 1.0% w/v to 4.0% w/v, 2.0% w/v to 3.5% w/v, 3.0% w/v to 3.5% w/v, 2.5% w/v to 4.0% w/v, 3.0% w/v to 4.0% w/v, 2.5% w/v to 3.0% w/v, and 3.0% w/v 5.0% w/v.

In one embodiment, the odor eliminating composition of the presently disclosed technology may include one or more antioxidants. As a non-limiting example, the odor eliminating composition may include at least 1, 2, 3, 4, 5, 6, 7, 8, 9, and at least 10 antioxidants. An antioxidant may include any substance that inhibits oxidation and/or reduces oxidized substances, including, but not limited to, Vitamin C, Vitamin E, reduced glutathione, argan oil, coconut oil, shea butter, green tea extract, any combination thereof, and/or the like. In some embodiments, the one or more antioxidants includes only Vitamin C and reduced glutathione. In some embodiments, the one or more antioxidants includes only reduced glutathione. In some embodiments, the one or more antioxidants includes only Vitamin C, Vitamin E, and reduced glutathione.

Optionally, the one or more antioxidants may be present in at least 0.1% to 5.0% w/v. The one or more antioxidants may be at least 0.1% w/v, including, but not limited to, 0.1, 0.2, 0.3, 0.4, 0.5, 0.6, 0.7, 0.8, 0.9, 1.0, 1.1, 1.2, 1.5, 2.0, 2.5, 3.0, 3.5, 4.0, 4.5, and at least 5.0% w/v. The one or more antioxidants may be no more than 5.0% w/v, including, but not limited to, no more than 5.0, 4.5, 4.0, 3.5, 3.0, 2.5, 2.0, 1.5, 1.2, 1.1, 1.0, 0.9, 0.8, 0.7, 0.6, 0.5, 0.4, 0.3, 0.2 and no more than 0.1% w/v. Any combination of the lower and upper limits is within the scope of the present disclosure, including, but not limited to, 0.1% w/v to 0.6% w/v, 0.2% w/v to 0.8% w/v, 0.3% w/v to 0.5% w/v, 0.4% w/v to 0.5% w/v, 0.3% w/v to 1.0% w/v, 0.3% w/v to 0.7% w/v, 0.3% w/v to 4.0% w/v, 2.0% w/v to 4.0% w/v, 1.0% w/v to 5.0% w/v, and 0.2% w/v 1.1% w/v.

Optionally, the one or more antioxidants may include 0.05% w/v to 0.4% w/v reduced glutathione. The reduced glutathione may include at least 0.05% w/v, including, but not limited to, at least 0.05, 0.075, 0.10, 0.125, 0.15, 0.175, 0.20, 0.25, 0.30, 0.35, and at least 0.40% w/v reduced glutathione. The reduced glutathione may include no more than 0.40% w/v reduced glutathione, including, but not limited to, 0.40, 0.35, 0.30, 0.25, 0.20, 0.175, 0.15, 0.125, 0.10, 0.075, and no more than 0.05% w/v. Any combination of the lower and upper limits is within the scope of the present disclosure, including, but not limited to, 0.05% w/v to 0.175% w/v, 0.125% w/v to 0.20% w/v, 0.20% w/v to 0.25% w/v, 0.15% w/v to 0.25% w/v, 0.175% w/v to 0.30% w/v, and 0.175% w/v to 0.35% w/v.

Optionally, the one or more antioxidants may include 0.05% w/v to 0.4% w/v Vitamin C. The Vitamin C may include at least 0.05% w/v, including, but not limited to, at least 0.05, 0.075, 0.10, 0.125, 0.15, 0.175, 0.20, 0.25, 0.30, 0.35, and at least 0.40% w/v Vitamin C. The Vitamin C may include no more than 0.40% w/v Vitamin C, including, but not limited to, 0.40, 0.35, 0.30, 0.25, 0.20, 0.175, 0.15, 0.125, 0.10, 0.075, and no more than 0.05% w/v. Any combination of the lower and upper limits is within the scope of the present disclosure, including, but not limited to, 0.05% w/v to 0.175% w/v, 0.125% w/v to 0.20% w/v, 0.20% w/v to 0.25% w/v, 0.15% w/v to 0.25% w/v, 0.175% w/v to 0.30% w/v, and 0.175% w/v to 0.35% w/v.

Optionally, the one or more antioxidants may include 0.05% w/v to 0.4% w/v Vitamin E. The Vitamin E may include at least 0.05% w/v, including, but not limited to, at least 0.05, 0.075, 0.10, 0.125, 0.15, 0.175, 0.20, 0.25, 0.30, 0.35, and at least 0.40% w/v Vitamin E. The Vitamin E may include no more than 0.40% w/v Vitamin E, including, but not limited to, 0.40, 0.35, 0.30, 0.25, 0.20, 0.175, 0.15, 0.125, 0.10, 0.075, and no more than 0.05% w/v. Any combination of the lower and upper limits is within the scope of the present disclosure, including, but not limited to, 0.05% w/v to 0.175% w/v, 0.125% w/v to 0.20% w/v, 0.20% w/v to 0.25% w/v, 0.15% w/v to 0.25% w/v, 0.175% w/v to 0.30% w/v, and 0.175% w/v to 0.35% w/v.

In one embodiment, the odor eliminating composition of the presently disclosed technology may include one or more pH stabilizers. As a non-limiting example, the odor eliminating composition may include at least 1, 2, 3, 4, and at least 5 pH stabilizers. A pH stabilizer may include any compound that helps maintain a desired pH level, including, but not limited to, L-arginine, lactic acid, sodium bicarbonate, potassium bicarbonate, magnesium hydroxide, magnesium lactate, citric acid, anhydrous citric acid, acetic acid, sodium acetate, sodium citrate, sodium citrate dihydrate, magnesium gluconate, aluminum hydroxide, aluminum hydroxide/sodium bicarbonate precipitate, a mixture of an amino acid and a buffer, a mixture of an acid salt and an amino acid and a buffer, a mixture of aluminum glycinate and a buffer, a mixture of an alkali salt of an amino acid and a buffer, sodium carbonate, sodium tartrate, sodium polyphosphate, potassium polyphosphate, sodium pyrophosphate, potassium pyrophosphate, disodium hydrosodium acetate, potassium metaphosphate, magnesium oxide, magnesium carbonate, magnesium silicate, calcium acetate, calcium glycerophosphate, calcium chloride, calcium hydroxide, calcium lactate, calcium carbonate, calcium bicarbonate, other calcium salts, combinations thereof, and the like.

Optionally, the one or more pH stabilizers may be present in at least 0.1% to 1.5% w/v. The one or more pH stabilizers may be at least 0.1% w/v, including, but not limited to, 0.1, 0.2, 0.3, 0.4, 0.5, 0.6, 0.7, 0.8, 0.9, 1.0, 1.25, and at least 1.5% w/v. The one or more pH stabilizers may be no more than 1.5% w/v, including, but not limited to, no more than 1.5, 1.25, 1.0, 0.9, 0.8, 0.7, 0.6, 0.5, 0.4, 0.3, 0.2 and no more than 0.1% w/v. Any combination of the lower and upper limits is within the scope of the present disclosure, including, but not limited to, 0.1% w/v to 0.8% w/v, 0.3% w/v to 0.7% w/v, 0.3% w/v to 0.6% w/v, 0.4% w/v to 0.7% w/v, 0.4% w/v to 0.8% w/v, 0.5% w/v to 0.7% w/v, 0.5% w/v to 1.25% w/v, and 0.5% w/v 1.0% w/v.

The odor eliminating composition of the presently disclosed technology may have a pH of at least 6.0, including, but not limited to, at least 6.0, 6.25, 6.5, 6.75, 7.0, 7.25, 7.50, 7.75 and at least 8.0. The pH may be no more than 8.0, including, but not limited to, no more than 8.0, 7.75, 7.5, 7.25, 7.0, 6.75, 6.5, 6.25 and no more than 6.0. Any combination of the lower and upper limits is within the scope of the present disclosure, including, but not limited to, a pH of 6.0 to 8.0, 6.25 to 7.50, 6.75 to 7.25, 6.50 to 7.25, 6.50 to 8.0, 6.75 to 7.50, and 6.5 to 7.5.

The odor eliminating composition of the presently disclosed technology may include one or more solvents, including, but not limited to, water, purified water, distilled water, or any solvent known in the art to achieve the desired clarity, viscosity, compatibility with excipients, chemical inertness, odor, and/or color. The solvent may be added to the formulation quantum satis to achieve the desired composition.

Optionally, the solvent may be present in at least 40% w/v to 85% w/v, including but not limited to, 40, 45, 50, 55, 60, 65, 70, 75, 80, and least 85% w/v. The solvent may be no more than 85% w/v, including, but not limited to, no more 85, 80, 75, 70, 65, 60, 55, 50, 45, and no more than 40% w/v. Any combination of the lower and upper limits is within the scope of the present disclosure, including, but not limited to, 40% w/v to 70% w/v, 50% w/v to 65% w/v, 60% w/v to 65% w/v, 60% w/v to 70% w/v, 50% w/v to 80% w/v, 50% w/v to 75% w/v, and 60% w/v to 75% w/v.

The odor eliminating composition of the presently disclosed technology may optionally include one or more fragrance. Some components discussed above may additionally serve as a fragrance, including, but not limited to, lavender, rosemary, lemon, and the like. In some embodiments, the odor eliminating composition may not have a detectable fragrance.

The odor eliminating composition of the presently disclosed technology may be “substantially free”, “essentially free” or “completely free” of impurities. As used herein, “substantially free” or “essentially free” refers to the presence in trace amounts or less. If present in trace amounts, impurities make no technical contribution to the composition.

In one embodiment, the odor eliminating composition of the presently disclosed technology may be in the form of a shampoo, body wash, fabric cleaner, or any other composition capable of being used to wash an animal or article (e.g., clothing). Optionally, the odor eliminating composition may be in the form of a conditioner. The conditioner may optionally include a fragrance. Generally, the odor eliminating conditioner should be applied after the odor eliminating shampoo. The odor eliminating conditioner may include additional components which influence the appearance, moisture, thickness, and general manageability of hair.

In some embodiments, the odor eliminating composition of the presently disclosed technology includes 13% w/v to 25% w/v one or more of a surfactant, 1% w/v to 11% w/v one or more of an emulsifier, 1.0% w/v to 5.0% w/v one or more of a humectant, 1.0% w/v 10.0% w/v one or more of an oil, 1.0% w/v to 6.0% w/v one or more of a preservative, 0.1% w/v to 5.0% w/v one or more of an antioxidant, 0.1% w/v to 1.5% w/v one or more of a pH stabilizer, and 40% w/v to 85% w/v one or more of a solvent.

In some embodiments, the odor eliminating composition of the presently disclosed technology includes 17% w/v to 21% w/v one or more of a surfactant, 5% w/v to 7% w/v one or more of an emulsifier, 1.4% w/v to 1.8% w/v one or more of a humectant, 5.0% w/v 6.0% w/v one or more of an oil, 2.5% w/v to 4.0% w/v one or more of a preservative, 0.3% w/v to 0.5% w/v one or more of an antioxidant, 0.5% w/v to 0.7% w/v one or more of a pH stabilizer, and 60% w/v to 70% w/v one or more of a solvent.

The odor eliminating composition of the presently disclosed technology may be used to eliminate odor from an animal secretion, including, but not limited to, skunk spray. The odor eliminating composition may be configured to breakdown thiol compounds responsible for skunk odor through one or more thiol-disulfide exchange reactions and redox processes.

The present disclosure provides methods of eliminating odor from skunk spray. Once an animal is sprayed by a skunk, the odor eliminating composition is applied to the entire body or at least parts of the body of the sprayed animal. Then, in one optional embodiment, the odor eliminating composition is allowed to sit for at least 3 to 5 minutes. After at least 3 to 5 minutes, the sprayed animal is washed and the odor eliminating composition is rinsed off along with the odor-causing compounds. Optionally, an odor eliminating conditioner may be applied after the first washing step. Optionally, the sprayed animal may be washed at least a second time, including, but not limited to a second, third, fourth, fifth, sixth, seventh, eighth, ninth, and tenth time with the odor eliminating composition.

The present disclosure provides methods of eliminating odor of an animal secretion on articles, including, but not limited to, clothes, furniture, cars, towels, and fabric. Once the article includes odor from an animal secretion (e.g., skunk spray), the odor eliminating composition is applied to the entire article or at least parts of the article. Then the odor eliminating composition is allowed to sit, for example, for at least 3 to 5 minutes. After at least 3 to 5 minutes, the article is washed and the odor eliminating composition is rinsed off along with the odor-causing compounds. In one embodiment, the shelf-life at room temperature of the odor eliminating composition may be at least 6 months, including, without limitation, 6 to 9 months, to 6 to 12 months, 6 to 15 months, 6 to 18 months, 6 to 21 months, 6 to 24 months, 6 to 27 months, 6 to 30 months, 6 to 33 months, and 6 to 36 months. In a preferred embodiment, the shelf-life at room temperature is at least 12 months.

The odor eliminating composition of the presently disclosed technology is safe for human and other animal use. Any dermatological or toxicological effects are negligible, if any. The odor eliminating composition is not destructive to articles.

The present disclosure further provides kits that may be used to practice the present technology. The kit may include at least one odor eliminating composition or at least one component of the odor eliminating composition described herein. In some embodiments, a kit includes odor eliminating compositions or any of the variations described herein, within a container, including, but not limited to, a plastic bottle, a glass bottle, and/or the like. In some embodiments, a kit includes a concentrated odor eliminating composition wherein a user is required to add water to achieve a desired overall concentration prior to application to a sprayed animal or article.

Any methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

In this description and in the appended claims, use of the singular forms “a”, “an”, and “the” includes the plural and plural encompasses singular, unless specifically stated otherwise. For example, although reference is made herein to “an” antioxidant, or “the” oil, one or more of any of these components and/or any other components described herein may be used.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Various embodiments may be described with reference to one or more exemplary implementations. As used herein, the term “exemplary” means “serving as an example, instance, or illustration,” and should not necessarily be construed as preferred or advantageous over other variations of the compositions and embodiments disclosed herein. “Optional” or “optionally” means that the subsequently described event or circumstance may or may not occur, and that the description includes instances where the implementation occurs and instances where it does not.

As used herein, the term “shelf-life” refers to the time period over which the product maintains its potency, efficacy and physical characteristic within acceptable limits.

The word “comprising” and forms of the word “comprising”, as used in this description and in the claims, does not limit the present disclosure to exclude any variants or additions. Additionally, although the present disclosure has been described in terms of “comprising”, the processes, materials, and compositions detailed herein may also be described as consisting essentially of or consisting of. For example, while certain embodiments of the present disclosure have been described in terms of an odor eliminating composition, an odor eliminating composition “consisting essentially of” or “consisting of” certain components is also within the present scope. In this context, “consisting essentially of” means that any additional components will not materially affect the efficacy of the liquid formulation or method of use thereof.

Moreover, other than in the examples, or where otherwise indicated, all numbers expressing, for example, quantities of ingredients used in the specification are to be understood as being modified in all instances by the term “about”. Accordingly, unless indicated to the contrary, the numerical parameters set forth in the following specification are approximations that may vary depending upon the desired properties to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Thus, the term “about” when used before a numerical designation, e.g., temperature, time, amount, and concentration, including a range, indicates approximations which may vary by ±10%, ±5%, or ±1%.

Notwithstanding that the numerical ranges and parameters setting forth the broad scope of the present disclosure are approximations and reported as precisely as possible, any numerical value inherently contains certain errors necessarily resulting from the standard variation found in their respective testing measurements. When ranges are given, any endpoints of those ranges and/or numbers within those ranges can be combined within the scope of the present disclosure.

As used herein, “animal” includes, any human or nonhuman animal, including, but not limited to, dogs, cats, nonhuman primates, cattle, sheep, pigs, horses, cows, rodents, chickens, amphibians, reptiles, other pets, other vertebrates, and the like.

Unless otherwise defined, all technical and scientific terms used herein have the same meaning as commonly understood by one of ordinary skill in the art to which the present disclosure belongs.

In the description, certain details are set forth to provide a better understanding of various embodiments of the methods disclosed herein. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While particular embodiments have been described, it would be obvious to those skilled in the art that various other changes and modifications may be made without departing from the spirit and scope of the present disclosure. Those skilled in the art will recognize or be able to ascertain using no more than routine experimentation, numerous equivalents to the specific apparatuses and methods described herein, including alternatives, variants, additions, deletions, modifications and substitutions. This application including the appended embodiments is therefore intended to cover all such changes and modifications that are within the scope of this application.

While compositions and methods are described herein, those skilled in the art recognize that the compositions and methods of the presently disclosed technology are not limited to the embodiments or drawings described. Rather, the presently disclosed technology covers all modifications, equivalents and alternatives falling within the spirit and scope of the appended claims. Any headings used herein are for organizational purposes only and are not meant to limit the scope of the description or the claims.

As used herein, the word “may” and “can” is used in a permissive sense (i.e., meaning having the potential to) rather than the mandatory sense (i.e., meaning must). The terminology includes the words noted above, derivatives thereof and words of similar import.

### EXAMPLES

Example 1: Six (6) 20-gram hair samples were collected from a mannequin model to test the efficacy of the odor eliminating composition in eliminating skunk spray odor. Hair only served as a control group (Sample 1).

The remaining five hair samples were treated with 15 mL of concentrated thiol oil, wherein hair samples were stirred and given five (5) minutes to rest to allow the hair to absorb the thiol oil. After the oil absorption, 10 mL of the odor eliminating composition of the present disclosure was added to three (3) of the hair samples (Samples 3, 4, & 5), while one (1) hair sample served as a control group for odor (Sample 2). One (1) hair sample was treated with a combination of hydrogen peroxide, baking soda, and liquid dish soap (Sample 6).

Samples 1 and 2 were not washed and remained as control groups. Sample 3 was washed one (1) time with the odor eliminating composition, allowed to sit for five (5) minutes, rinsed with 50 mL of water, and pat dried. Sample 4 was washed one (1) time with the odor eliminating composition, allowed to sit for five (5) minutes, rinsed with 50 mL of water, pat dried, washed a second time with the odor eliminating composition, allowed to sit for five (5) minutes, rinsed with 50 mL of water and pat dried a second time. Sample 5 was washed one (1) time with the odor eliminating composition, allowed to sit for five (5) minutes, rinsed with 50 mL of water, pat dried, washed a second time with the odor eliminating composition, allowed to sit for five (5) minutes, rinsed with 50 mL of water, washed with an odor eliminating composition having a fragrance, rinsed with 50 mL and pat dried. Sample 6 was washed with the combination of baking soda, hydrogen peroxide, and soap, allowed to sit for five (5) minutes, rinsed with 50 mL of water, and pat dried. Each sample was placed within a container, and the container was exchanged for a new container after each wash to ensure there was no lingering skunk odor sticking to the container.

Each sample was assessed for odor levels by fifty (50) randomly selected participants in a single blind study. Participants rated the odor on a scale of 0 through 10, wherein 0 represented no detectable odor and 10 represented intense and foul presence of odor. Prior to each participant inhaling a scent and between each sample, participants were given a full container of coffee beans to smell. After smelling the coffee beans, each participant was asked to cup their hands in front of their nose and smell their own skin in an attempt to neutralize the sense of smell between assessments. The results are present in Table 1 below.

TABLE 1
Statistical Analysis

| Sample | Mean | Std. Dev.* | Std. Error |
|---|---|---|---|
| 1 | 0.06 | 0.3136 | 0.0444 |
| 2 | 9.56 | 0.9293 | 0.314 |
| 3 | 4.1 | 1.9086 | 0.2699 |
| 4 | 2.1 | 1.7053 | 0.2412 |
| 5 | 0.7 | 1.4033 | 0.1985 |
| 6 | 6.74 | 1.9568 | 0.2767 |

*Std. Dev. is standard deviation.

A standard ANOVA analysis was performed, the F-Stat and P-value between groups was 308.15 and 0, respectively. The results indicate variance between the samples is significantly greater than the variance within the samples, suggesting that at least one sample mean is different from the others.

More than one wash with the odor eliminating composition resulted in a decreased odor. Overall, the results indicate that the odor eliminating composition of the present disclosure is effective in neutralizing and removing skunk odor compared to the control and prior art methods

What is claimed is:

1. An odor eliminating composition, comprising:
13% w/v to 25% w/v one or more of a surfactant;
1.0% w/v to 11% w/v one or more of an emulsifier;
1.0% w/v to 5.0% w/v one or more of a humectant;
1.0% w/v 10.0% w/v one or more of an oil;
1.0% w/v to 6.0% w/v one or more of a preservative;
0.1% w/v to 5.0% w/v one or more of an antioxidant;
0.1% w/v to 1.5% w/v one or more of a pH stabilizer; and
40% w/v to 85% w/v one or more of a solvent,
wherein the one or more antioxidants comprises reduced glutathione, and
wherein the one or more oils comprises garlic oil.

2. The odor eliminating composition of claim 1, wherein the odor eliminating composition comprises 17% w/v to 21% w/v one or more of a surfactant, 5% w/v to 7% w/v one or more of an emulsifier, 1.4% w/v to 1.8% w/v one or more of a humectant, 5.0% w/v 6.0% w/v one or more of an oil, 2.5% w/v to 4.0% w/v one or more of a preservative, 0.3% w/v to 0.5% w/v one or more of an antioxidant, 0.5% w/v to 0.7% w/v one or more of a pH stabilizer, and 60% w/v to 70% w/v one or more of a solvent.

3. The odor eliminating composition of claim 1, wherein the one or more of an antioxidant further comprises Vitamin C.

4. The odor eliminating composition of claim 3, wherein the one or more of an antioxidant further comprises Vitamin E.

5. The odor eliminating composition of claim 3, wherein the Vitamin C is present in an amount of 0.15% w/v to 0.25% w/v.

6. The odor eliminating composition of claim 1, wherein the garlic oil is present in an amount of 0.005% w/v to 0.040% w/v.

7. The odor eliminating composition of claim 6, wherein the garlic oil is present in an amount of 0.0125% w/v to 0.0175% w/v.

8. The odor eliminating composition of claim 1, wherein the reduced glutathione is present in an amount of 0.05% w/v to 0.40% w/v.

9. The odor eliminating composition of claim 8, wherein the reduced glutathione is present in an amount of 0.15% w/v to 0.25% w/v.

10. The odor eliminating composition of claim 1, wherein the pH is 6.0 to 8.0.

11. The odor eliminating composition of claim 1, wherein the composition is a shampoo or a conditioner.

12. An odor eliminating composition, comprising:
one or more of a surfactant, one or more of an emulsifier, one or more of a humectant, one or more of an oil, one or more of a preservative, one or more of an antioxidant, one or more of a pH stabilizer, one or more of a solvent,
wherein the one or more of an antioxidant comprises reduced glutathione
wherein the one or more of an oil comprises garlic oil, and
wherein the garlic oil comprises one or more of aliin, allicin, diallyl sulfide, diallyl disulfide, diallyl trisulfide, S-allylcysteine, S-allylmercaptocystein, allinase, peroxidase, myrosinase, arginine, selenium, germanium, and tellurium.

13. The odor eliminating composition of claim 12, wherein the one or more of an antioxidant further comprises Vitamin C.

14. A method of eliminating odor sprayed by a skunk, the method comprising:
applying an odor eliminating composition to an animal or an article, the odor eliminating composition comprising one or more of a surfactant, one or more of an emulsifier, one or more of a humectant, one or more of an oil, one or more of a preservative, one or more of an antioxidant, one or more of a pH stabilizer, one or more of a solvent, wherein the one or more of an antioxidant comprises reduced glutathione, wherein the one or more of an oil comprises garlic oil, and wherein the garlic oil comprises one or more of aliin, allicin, diallyl sulfide, diallyl disulfide, diallyl trisulfide, S-allylcysteine, S-allylmercaptocystein, allinase, peroxidase, myrosinase, arginine, selenium, germanium, and tellurium;
allowing the odor eliminating composition to remain on the animal or the article for at least three to five minutes; and
rinsing off the odor eliminating composition of the animal or the article.

15. The method of claim 14, wherein the method is repeated at least a second time.

16. The method of claim 14, further comprising applying an odor eliminating conditioner comprising one or more of an antioxidant, one or more of a solvent, and one or more of a fragrance, wherein the one or more of an oil comprises garlic oil, and wherein the garlic oil comprises one or more of aliin, allicin, diallyl sulfide, diallyl disulfide, diallyl trisulfide, S-allylcysteine, S-allylmercaptocystein, allinase, peroxidase, myrosinase, arginine, selenium, germanium, and tellurium.

17. The method of claim 14, wherein the odor eliminating composition further comprises Vitamin C.

18. The method of claim 14, wherein the odor eliminating composition is configured to breakdown thiol compounds responsible for skunk odor through one or more thiol-disulfide exchange reactions and redox processes.
